(19) United States
(12) Patent Application Publication
(10) Pub. No.: US 2023/0326575 A1
(43) Pub. Date: Oct. 12, 2023

(54) SYSTEM AND METHOD FOR PROVIDING FERTILITY ENHANCING DIETARY RECOMMENDATIONS IN INDIVIDUALS WITH LOW TESTOSTERONE

(71) Applicant: SOCIETE DES PRODUITS NESTLE S.A., Vevey (CH)

(72) Inventors: MYRIAM AFEICHE ZEHIL, Lausanne 26 (CH); FLORENT DUDAN, St-Sulpice (CH); FABIO MAINARDI, Lausanne (CH); ROKO PLESTINA, Morges (CH); IAROSLAVA SEMENOVA, Lausanne (CH)

(21) Appl. No.: 17/997,082

(22) PCT Filed: Apr. 27, 2021
(86) PCT No.: PCT/EP2021/060886

§ 371 Date: Oct. 25, 2022

(30) Foreign Application Priority Data
Apr. 29, 2020 (EP) 20172017.4

(51) Int. Cl. G16H 20/60; G16H 50/30; G16H 50/20; A61K 31/593; A61K 31/568; A61K 33/06; A61K 31/5685; A61K 33/30; A61K 36/185; A61K 36/48; A61K 31/519; A61P 5/26

(52) U.S. Cl. CPC G16H 20/60; G16H 50/30; G16H 50/20; A61K 31/593; A61K 31/568; A61K 33/06; A61K 31/5685; A61K 33/30; A61K 36/185; A61K 36/48; A61K 31/519; A61P 5/26; G16H 20/10

(57) ABSTRACT

The present invention presents new and innovative methods and systems for personalized, real-time diet and lifestyle recommendations for users that are seeking to improve their own fertility. In a preferred embodiment, the present invention relates to novel methods and systems for personalized dietary recommendations for improving fertility in individuals, especially men, with low testosterone.

### FIELD OF THE INVENTION

The present invention presents new and innovative methods and systems for personalized, real-time diet and lifestyle recommendations for users that are seeking to improve their own fertility.

In a preferred embodiment, the present invention relates to novel methods and systems for personalized dietary recommendations for improving fertility in individuals, especially men, with low testosterone.

### BACKGROUND TO THE INVENTION

Testosterone is a key androgenic hormone regulating sperm production and the development of male reproductive tissues such as the testes and prostate, as well as promoting secondary sexual characteristics such as increased muscle and bone mass, and the growth of body hair.

Hypogonadism is the condition of low testosterone which occurs when an adult male's testosterone level falls below the normal range of about 270 to 1,070 nanograms per deciliter (ng/dL). Low testosterone levels can directly affect fertility by causing decreased sperm production and indirectly affect fertility by reducing libido and causing erectile dysfunction.

There may be inter-individual differences in normal testosterone level between adult men. For example, it is known that testosterone levels decrease with increasing age from around age 30 years onwards. Such a decrease in testosterone levels is considered to be a normal part of the ageing process. It may only become a problem as men choose to delay the age at which they wish to conceive a child.

In addition to increasing age, other risk factors for low testosterone levels include: obesity, testicular injury and scrotal overheating, excessive alcohol consumption, chronic narcotic use, marijuana abuse, diabetes, and cancer treatments such as chemotherapy. Low testosterone may also be caused by testicular cancer, early or late delayed puberty and kidney disease.

Lifestyle changes such as keeping the testicles and scrotum cool by avoiding overheating such as in saunas and hot tubs, stopping smoking and losing weight may improve testosterone levels.

Testosterone replacement therapy (TRT) may be prescribed for individuals suffering from low testosterone. However, a critical side effect of testosterone replacement therapy (TRT) is infertility. TRT can decrease sperm production because it also lowers the level of the follicle-stimulating hormone (FSH), which is responsible for stimulating sperm production. In many cases, the infertility caused by testosterone treatment can be reversed. Therefore, men seeking to conceive with their partner are advised not use medications to treat their low testosterone.

Therefore, there is a need to find alternative ways for increasing testosterone levels in order to improve fertility in men with low testosterone without decrease in sperm production and infertility.

At present, dietary recommendations for increasing low testosterone levels are often too general as studies on fertility and diet often investigate only single nutrients at a time or food groups without putting them in the context of an entire diet over a day or entire meal and they do not provide recommended intake amounts to be consumed per day or for each specific, different medical condition which affects fertility, such as low testosterone.

The present invention addresses the deficiencies in the state of the art by providing new and innovative methods and systems for personalized, real-time diet and lifestyle recommendations for individual users.

The dietary recommendations of the present invention may contribute to improving low testosterone-related symptoms such as decreased sperm production, reduced libido and erectile dysfunction.

In particular, the present invention addresses the specific condition of enhancing fertility and conception in individuals with low testosterone, by providing a novel, consolidated dietary recommendations which combine:
specific dietary components recommended to be consumed daily
specific dietary intake amounts
specific recommendations on avoiding certain dietary components
specific recommendations on lifestyle components.

### SUMMARY OF THE INVENTION

The present invention presents novel and innovative methods and systems for personalized, real-time diet and lifestyle recommendations for users that are seeking to improve their own fertility.

In several embodiments, a method and system is provided that includes requesting and receiving a plurality of user attributes, comparing the plurality of user attributes to a corresponding plurality of evidence-based fertility benchmarks, determining a plurality of fertility support opportunities based on the plurality of user attributes and the comparison to the corresponding plurality of evidence-based fertility benchmarks, identifying a plurality of fertility enhancing recommendations based on the plurality of fertility support opportunities, and presenting at least one of the plurality of fertility enhancing recommendations.

In a preferred embodiment of the invention, the system and method present fertility enhancing recommendations are for increasing fertility in a male individual with low testosterone. Low testosterone levels are considered to be below 270 nanograms per deciliter (ng/dL) in a sample of semen.

In another preferred embodiment of the invention, the dietary recommendations of the present invention may contribute to improving low testosterone-related symptoms such as decreased sperm production, reduced libido and erectile dysfunction.

### DESCRIPTION OF FIGURES

FIG. 1 illustrates a system according to an embodiment of the present invention.

FIG. 2 illustrates system components according to exemplary embodiments of the present invention.

FIG. 3 illustrates system components according to an exemplary embodiment of the present invention.

FIG. 4 illustrates an example plurality of dietary recommendations for a user according to an exemplary embodiment of the present invention.

FIG. 5 illustrates a method according to an embodiment of the present invention.

FIGS. 6A and 6B illustrate a method according to an embodiment of the present invention.

### DETAILED DESCRIPTION OF THE INVENTION

To promote user fertility, it may be useful to provide a customized diet and lifestyle plan for users interested enhancing fertility. Therefore, a customized, integrated approach is necessary to provide maximum benefit to enhance chances of fertility and conception.

In several embodiments of the invention, the customized diet and lifestyle plan is individualized to male users with hypogonadism or low testosterone levels.

### Hypogonadism

There are two basic types of hypogonadism: primary and secondary hypogonadism.

Primary hypogonadism originates primarily from testicular failure.

Secondary hypogonadism originates in the hypothalamus or the pituitary gland which send signals from the brain to the testicles to produce testosterone. The hypothalamus produces gonadotropin-releasing hormone, which signals the pituitary gland to make follicle-stimulating hormone (FSH) and luteinizing hormone (LH). Luteinizing hormone then signals the testes to produce testosterone.

Either type of hypogonadism can be caused by an inherited trait (congenital) or as a result of an injury or infection that happens later in life (acquired). It is also possible for primary and secondary hypogonadism to both occur.

### Primary Hypogonadism

Common causes of primary hypogonadism are described below.

Klinefelter syndrome. This condition results from a congenital abnormality of the sex chromosomes, X and Y. A male normally has one X and one Y chromosome. In Klinefelter syndrome, two or more X chromosomes are present in addition to one Y chromosome.

The Y chromosome contains the genetic material that determines the sex of a child and related development. The extra X chromosome that occurs in Klinefelter syndrome causes abnormal development of the testicles, which in turn results in underproduction of testosterone.

Undescended testicles. Before birth, the testicles develop inside the abdomen and normally move down into their permanent place in the scrotum. Sometimes one or both of the testicles aren't descended at birth. This condition often corrects itself within the first few years of life without treatment. If not corrected in early childhood, it can lead to malfunction of the testicles and reduced production of testosterone.

Mumps orchitis. A mumps infection involving the testicles that occurs during adolescence or adulthood can damage the testicles, affecting the function of the testicles and testosterone production.

Hemochromatosis. Too much iron in the blood can cause testicular failure or pituitary gland dysfunction, affecting testosterone production.

Injury to the testicles. Because they're outside the abdomen, the testicles are prone to injury. Damage to normally developed testicles can cause hypogonadism. Damage to one testicle might not impair total testosterone production.

Cancer treatment. Chemotherapy or radiation therapy for the treatment of cancer can interfere with testosterone and sperm production. The effects of both treatments often are temporary, but permanent infertility may occur. Although many men regain their fertility within a few months after treatment, preserving sperm before starting cancer therapy is an option for men.

### Secondary Hypogonadism

In secondary hypogonadism, the testicles are normal but don't function properly due to a problem with the pituitary or hypothalamus. A number of conditions can cause secondary hypogonadism, including:

Kallmann's syndrome. This is an abnormal development of the area of the brain that controls the secretion of pituitary hormones (hypothalamus). This abnormality can also affect the ability to smell (anosmia) and cause red-green color blindness.

Pituitary disorders. An abnormality in the pituitary gland can impair the release of hormones from the pituitary gland to the testicles, affecting normal testosterone production. A pituitary tumor or other type of brain tumor located near the pituitary gland may cause testosterone or other hormone deficiencies. Also, treatment for a brain tumor, such as surgery or radiation therapy, can affect the pituitary gland and cause hypogonadism.

Inflammatory disease. Certain inflammatory diseases, such as sarcoidosis, histiocytosis and tuberculosis, involve the hypothalamus and pituitary gland and can affect testosterone production.

HIV/AIDS. HIV/AIDS can cause low levels of testosterone by affecting the hypothalamus, the pituitary and the testes.

Medications. The use of certain drugs, such as opiate pain medications and some hormones, can affect testosterone production.

Obesity. Being significantly overweight at any age might be linked to hypogonadism.

Normal aging. As men age, there is a slow, progressive decrease in testosterone production after about the age of 30 onwards. The rate varies greatly between individuals with age (Zirkin et al. 2012).

In any of the above conditions of primary or secondary hypogonadism, the methods and systems of the invention for providing individualized recommendations may be beneficial.

In several embodiments of the invention, the recommendations are personalized based on information from an individual regarding certain medical conditions or diseases and the current status of conception to compare with a historical evidence-based fertility database to generate recommended diet and lifestyle options that will help improve the patient's fertility based on the provided information.

An example system may be beneficial if it can provide user support throughout the entire conception journey, from the early stages of planning to the final stages of conception. As such, this example system would be useful should it provide constant, around the clock access to both virtual and personal fertility, lifestyle, nutrition, and exercise coaches. Furthermore, an example system may provide recommendations to manage anxiety, reduce stress, or provide specific supplementation, all of which are also linked to a user's fertility. The system may provide different recommendations for the male and female individuals in the couple that wish to conceive.

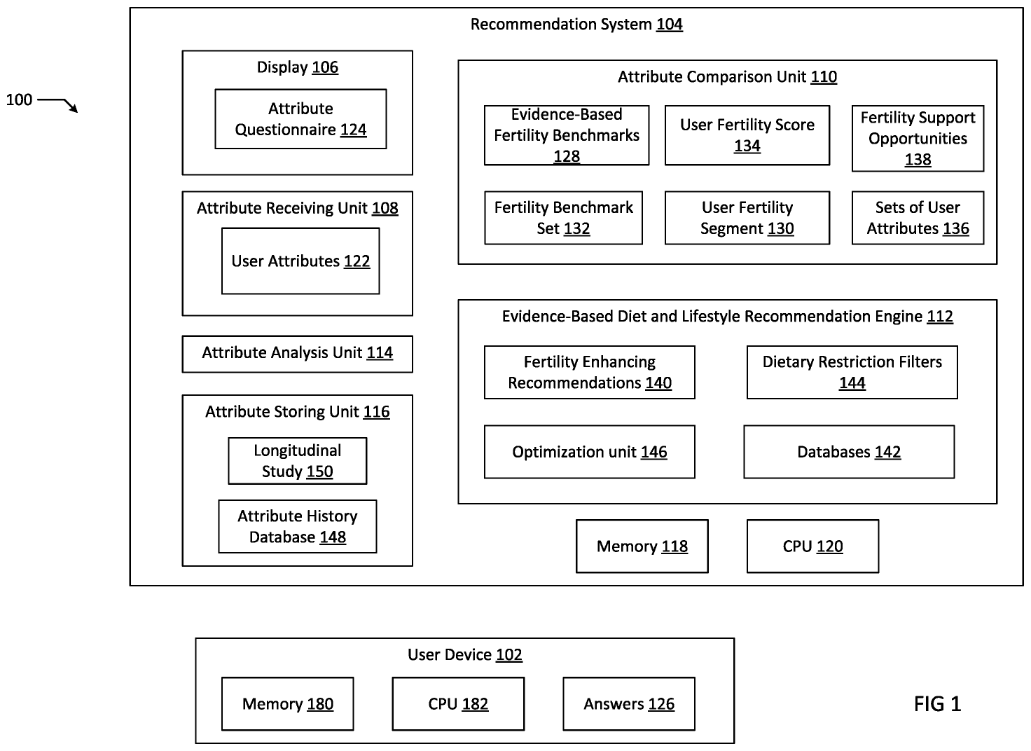

FIG. 1 illustrates a system 100 according to an embodiment of the present disclosure. The system 100 includes a user device 102 and a recommendation system 104. The user device 102 may be implemented as a computing device, such as a computer, smartphone, tablet, smartwatch, or other wearable through which an associated user can communicate with the recommendation system 104. The user device 102 may also be implemented as, e.g., a voice assistant configured to receive voice requests from a user and to process the requests either locally on a computer device proximate to the user or on a remote computing device (e.g., at a remote computing server).

The recommendation system 104 includes one or more of a display 106, an attribute receiving unit 108, an attribute comparison unit 110, an evidence-based diet and lifestyle recommendation engine 112, an attribute analysis unit 114, an attribute storing unit 116, a memory 118, and a CPU 120. Note, that in some embodiments, a display 106 may additionally or alternatively be located within the user device 102. In an example, the recommendation system 104 may be configured to receive a request for a plurality of fertility enhancing recommendations 140. For example, a user may install an application on the user device 102 that requires the user to sign up for a recommendation service. By signing up for the service, the user device 102 may send a request for the fertility enhancing recommendations 140. In a different example, the user may use the user device 102 to access a web portal using user-specific credentials. Through this web portal, the user may cause the user device 102 to request fertility enhancing recommendations from the recommendation system 104.

In another example, the recommendation system 104 may be configured to request and receive a plurality of user attributes 122. For example, the display 106 may be configured to present an attribute questionnaire 124 to the user. The attribute receiving unit 108 may be configured to receive the user attributes 122. In one example, the attribute receiving unit 108 may receive a plurality of answers 126 based on the attribute questionnaire 124, and based on the plurality of answers, determine the plurality of user attributes 122. For example, the attribute receiving unit 108 may receive answers to the attribute questionnaire 124 suggesting that the diet of the user is equivalent to the recommended dietary allowance (“RDA”) and then determine the user attributes 122 to be equivalent to the RDA. In another example, the user device attribute receiving unit 108 may directly receive the user attributes 122 from the user device 102.

In another example, the attribute receiving unit 108 may be configured to receive the test results of a home-test kit, the results of a standardized health test administered by a medical professional, the results of a self-assessment tool used by the user, or the results of any external or third party test. Based on the results from any of these tests or tools, the attribute receiving unit 108 may be configured to determine the user attributes 122. For example, this may be measurements of the nutrient levels in blood or urine of the user which may be compared to standardized nutrient levels. In the case of low testosterone, measurements of testosterone in the semen of the user may be compared to standardized testosterone levels and starting levels before the intervention to determine whether there are improvements in the level of testosterone over time.

The recommendation system 104 may be further configured to compare the plurality of user attributes 122 to a corresponding plurality of evidence-based fertility benchmarks 128. For example, the attribute comparison unit 110 may be configured to determine a user fertility segment 130.

In a preferred example, the user fertility segment could be much more specific. For example, the user fertility segment could be a user with low testosterone.

Furthermore, the attribute comparison unit 110 may be further configured to determine a fertility benchmark set 132 based on the user fertility segment 130. For example, if the attribute comparison unit 110 determines that a user falls into the low testosterone user fertility segment 130, based on the plurality of user attributes 122, the attribute comparison unit 110 may select a fertility benchmark set 132 that has been created and defined according to the specific needs of a user undergoing a particular medical treatment, such as hormonal treatment.

The comparison unit 110 may be further configured to select, from this determined fertility benchmark set 132, the evidence-based fertility benchmarks 128 and compare the now selected evidence-based fertility benchmarks 128 to each of the corresponding user attributes 122. For example, when the fertility benchmark set 132 has been determined, in response to the determination, the attribute comparison unit 110 may compare a user attribute 122 that represents the user's vitamin D intake to an evidence based fertility benchmark 128 that represents a benchmark vitamin D intake, determining whether the user is below, at, or above the benchmark vitamin D intake. Though this example is based on a concrete, numerical comparison, another example of a benchmark comparison may be qualitative and different depending on a person. For example, a user attribute 122 may indicate that the user is currently experiencing higher than normal levels of stress. An example benchmark related to a user stress level may indicate that an average or low level of stress is desired and thus, the user attribute 122 indicating a higher level of stress is determined to be below that of the benchmark. As different users experience differing levels of stress, even under the same circumstances, such a comparison requires a customized approach.

In addition, during the comparison from the prior example, the attribute comparison unit 110 may be configured to determine a user fertility score 134 based on the comparison between the evidence-based fertility benchmarks 128 and the user attributes 122. For example, the attribute comparison unit 110 may determine a user fertility score of 95/100 if the user attributes 122 very nearly meet all or most of the corresponding evidence-based fertility benchmarks 128. In another example, a score may be represented through lettering grades, symbols, or any other system of ranking that allows a user to interpret how well their current attributes rate amongst benchmarks. This user fertility score 134 may be presented through the display 106.

The recommendation system 104 may be further configured to determine a plurality of fertility support opportunities 138 based on the plurality of user attributes 122 and the comparison to the corresponding plurality of evidence-based fertility benchmarks 128. In one example, the attribute comparison unit 110 may determine fertility support opportunities 138 for every user attribute 122 that does not meet the corresponding evidence-based fertility benchmark. In this example, a corresponding evidence-based fertility benchmark 128 may require a user have an intake of 1000 mg/day of Vitamin D, whereas the user attribute may indicate the user is only receiving 500 mg/day of Vitamin D. Therefore, the attribute comparison unit 110 may determine an increase in Vitamin D intake to be a fertility support opportunity 138.

In another example, the attribute comparison unit 110 may be configured to identify a first set of user attributes 136 comprised of each of the plurality of user attributes 122 that are below the corresponding one of the plurality of evidence-based fertility benchmarks 128 as well as identify a second set of user attributes 136 comprised of each of the plurality of user attributes 122 that are greater than or equal to the corresponding evidence-based fertility benchmarks 128. While the first set of user attributes 136 is determined similarly to the above given example, the second set of user attributes 136 differs in that, although the associated user does not appear to have a deficiency, there may be opportunities to support fertility by recommending the user maintain current practices or opportunities to further improve upon them. Accordingly, the recommendation system 104 may determine opportunities to support fertility based on which attributes 122 populate either sets 136.

The recommendation system 104 may be further configured to identify a plurality of fertility enhancing recommendations 140 based on the plurality of fertility support opportunities 138. For example, the evidence-based diet and lifestyle recommendation engine 112 may be configured to be cloud-based. The recommendation engine 112 may comprise one or more of a plurality of databases 142, a plurality of dietary restriction filters 144, and an optimization unit 146. Based on the plurality of opportunities 138, the recommendation engine 112 may identify the plurality of fertility enhancing recommendations 140 according to the one or more of plurality of databases 142, the dietary restriction filters 144, and the optimization unit 146.

In another example, the recommendation system 104 may be configured to provide continuous recommendations, based on prior user attributes. For example, the recommendation system 104 may comprise, in addition to the previously discussed elements, an attribute storing unit 116 and an attribute analysis unit 114. The attribute storing unit 116 may be configured to, responsive to the attribute receiving unit 108 receiving the plurality of user attributes 122, add the received user attributes 122 to an attribute history database 148 as a new entry based on when the plurality of user attributes 122 were received. For example, if user attributes 122 are received by the attribute receiving unit 108 on a first day, the attribute storing unit 116 will add the received user attributes 122 to a cumulative attribute history database 148 noting the date of entry, in this case the first day. Later, if user attributes 122 are received by the attribute receiving unit 108 on a second day, e.g. the next day, the attribute storing unit 116 will also add these new attributes to the attribute history database 148, noting that they were received on the second day, while also preserving the earlier attributes from the first day.

This attribute analysis unit 114 may be configured to analyze the plurality of user attributes 122 stored within the attribute history database 148, wherein analyzing the stored plurality of user attributes 122 comprises performing a longitudinal study 150. Continuing the earlier example, the attribute analysis unit 114 may perform a longitudinal study of the user attributes 122 from each of the first day, the second day, and every other collection of user attributes 122 found within the attribute history database 148. The evidence based diet and lifestyle recommendation engine 112 may be further configured to generate a plurality of fertility enhancing recommendations 140 based on at least the stored user attributes 122 found within the attribute history database 148 and the analysis performed by the attribute analysis unit 114.

In an embodiment, the attribute analysis unit 114 is further configured to repeatedly analyze the plurality of user attributes 122 stored within the attribute history database 148 responsive to the attribute storing unit 116 adding a new entry to the attribute history database 148, essentially re-analyzing all of the data within the attribute history database 148 immediately after new user attributes 122 are received. Similarly, the evidence based diet and lifestyle recommendation engine 112 may be further configured to repeatedly generate the plurality of fertility enhancing recommendations 140 responsive to the attribute analysis unit 114 completing an analysis, thereby effectively generating new fertility enhancing recommendations 140 that consider all past and present user attributes 122 each time a new set of user attributes 122 is received.

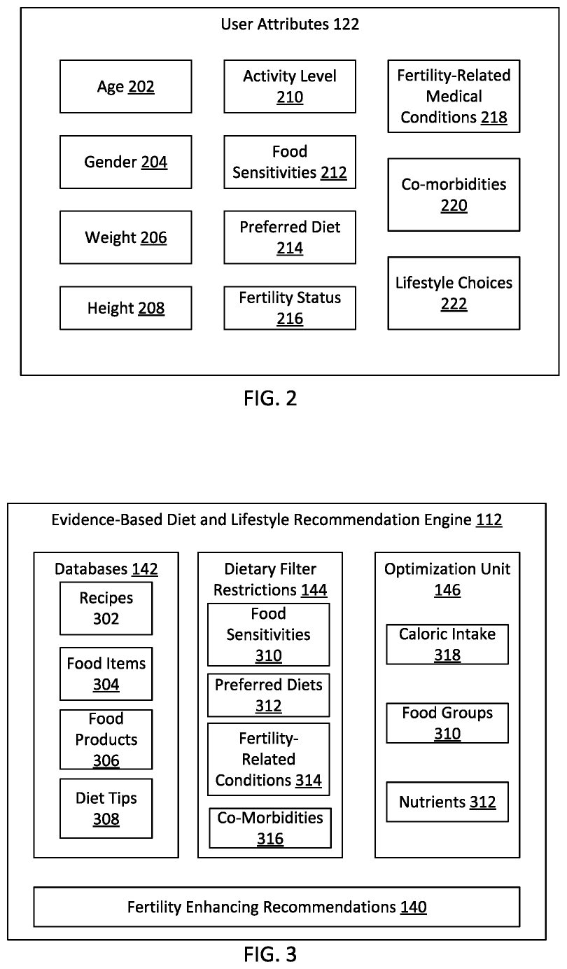

FIG. 2 illustrates an example database containing a plurality of user attributes 122. For example, the user attributes 122 may be populated by information regarding one or more of age 202, gender 204, weight 206, height 208, activity level 210, food sensitivities 212, preferred diet 214, fertility status 216, fertility-related medical conditions 218, co-morbidities 220, and lifestyle choices 222. Some examples of food sensitivities 212 include lactose, eggs, nuts, shellfish, soy, fish, and gluten sensitivities. Some non-limiting examples of a preferred diet 214 includes vegetarian, vegan, Mediterranean, kosher, halal, paleo, low carb, and low fat diets.

Some non-limiting examples of fertility-related medical conditions 218 include polycystic ovary syndrome, premature ovarian insufficiency, endometriosis, recurring pregnancy loss, undergoing IVF, semen abnormality, misuse of anabolic steroids and protein supplements, erectile dysfunction, hormonal imbalance, low testosterone, and prostate issues.

In a preferred embodiment, the fertility-related medical condition 218 is low testosterone.

Some non-limiting examples of co-morbidities 220 include diabetes, obesity, high blood pressure, high cholesterol, celiac, and heartburn. Some non-limiting examples off lifestyle choices 222 may include sleeping habits such as the typical hours of sleep per night, stress attributes such as the level of stress currently experienced by the user or typical levels of stress experienced, whether the user smokes, the number of alcoholic drinks typically consumed, exercise frequency, or any other lifestyle choices 222 that may have a bearing on fertility.

FIG. 3 illustrates an example embodiment of an evidence-based diet and lifestyle recommendation engine 112. In an example embodiment, the evidence-based diet and lifestyle recommendation engine 112 comprises a plurality of databases 142, a plurality of dietary filter restrictions 144, and an optimization unit 146. The plurality of databases 142 may include a database comprised of one or more of recipes 302, food items 304, food products 306, and diet tips 308. The dietary filter restrictions 144 may comprise filters for one or more of food sensitivities 310, preferred diets 312, fertility-related conditions 314, and co-morbidities 316. The optimization unit 146 may contain optimization rules based on one or more of caloric intake 318, food groups 310, and specific nutrients 312.

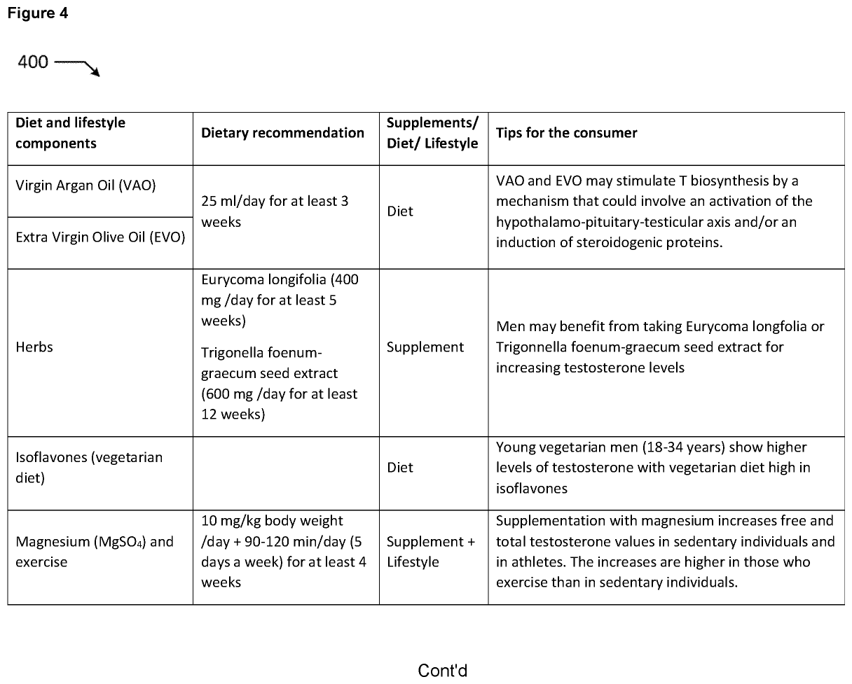

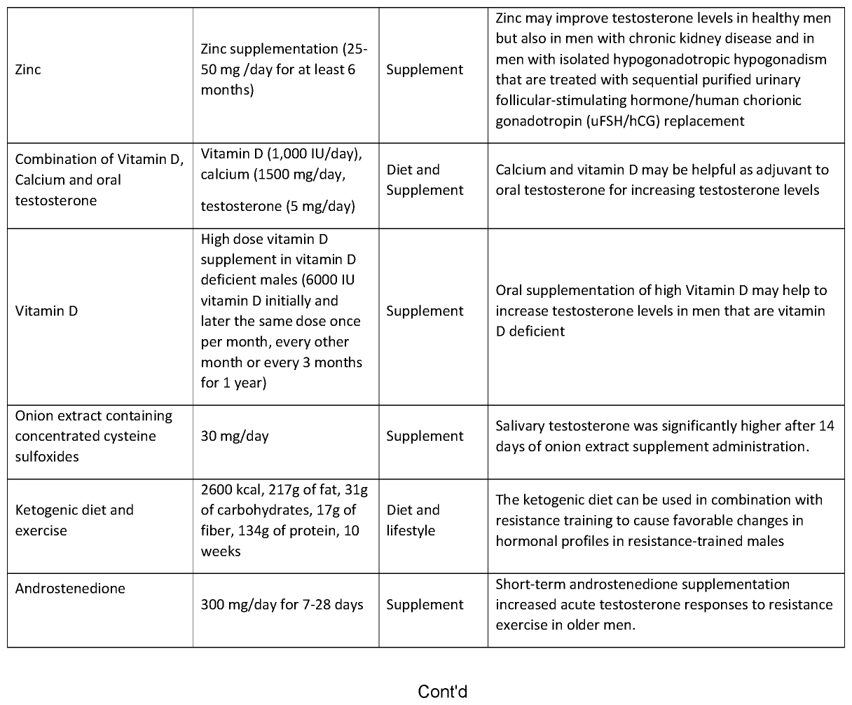

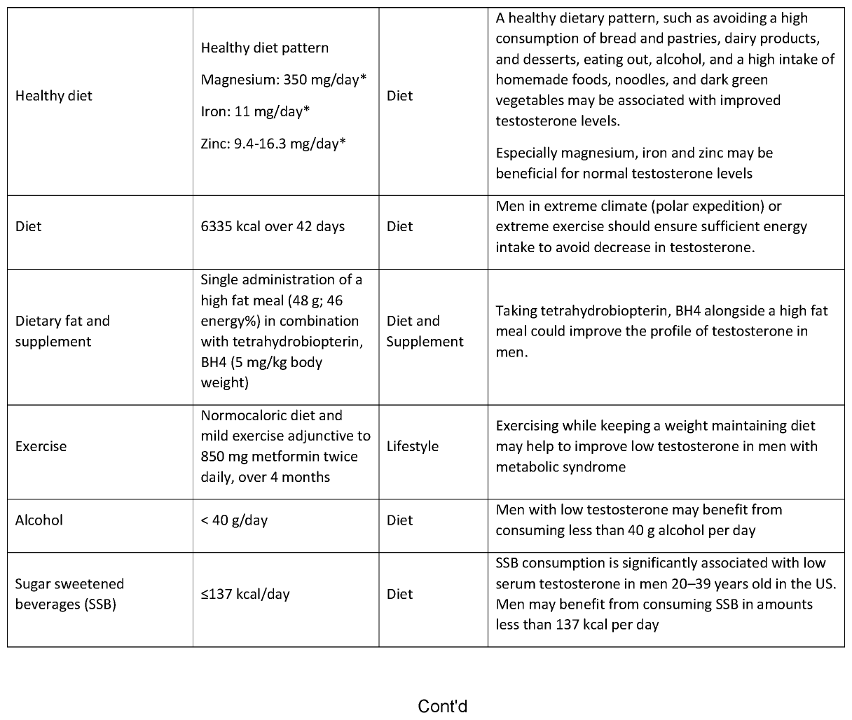

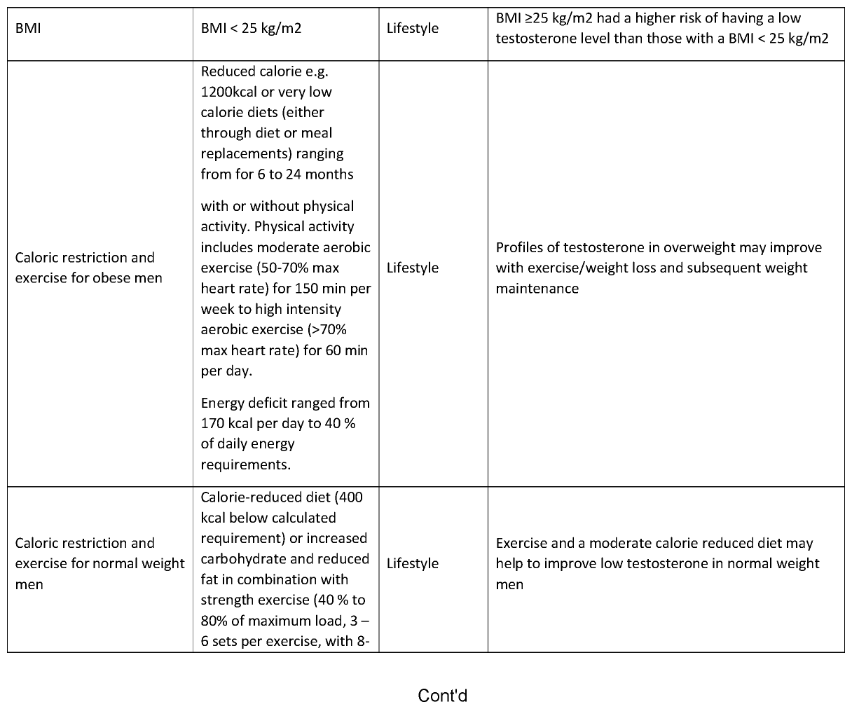

FIG. 4 illustrates an example plurality of dietary and lifestyle recommendations according to an exemplary embodiment of the present disclosure. This dietary recommendation example 400 details specific recommendations that may be presented to a user after a plurality of fertility enhancing recommendations 140 have been determined by the recommendation system 104. Specifically, example 400 details the fertility enhancing recommendations 140 as determined for a user that has a specific fertility-related medical condition 218. Specifically, example 400 represents the fertility enhancing recommendations as determined for a user with low testosterone.

Other recommendations 140 may be to simply avoid, or increase, consumption of a particular food item. Similarly, the recommendations 140 may include recommendations to consume certain substances moderately or to prefer one substance over another. Though there are many different types of recommendations 140 found within the example 400, one should appreciate that any type of qualitative or quantitative recommendation may be made regarding these food items and nutrients.

Furthermore, the recommendation system 400 may generate fertility enhancing recommendations 140 that include lifestyle changes, such as altering activity level, increasing the number of hours of rest per night, taking action to reduce stress, or similar lifestyle-affecting actions. For example, a high level of stress may negatively affect a user's fertility. Such stress may derive from the relationship between the partners actively trying to conceive. Some example fertility enhancing recommendations 140 may include suggestions for methods by which the couple may decrease tensions in the relationship in order to alleviate stress. In another example, the fertility enhancing recommendations 140 may include recommendations to increase the amount of time a user rests, including sleeping habit recommendations. These recommendations may range from general recommendations, such as instruction to get more sleep, to more detailed recommendations, including specific exercise routines, specific diets and recipes, or suggested dates for visits to a medical professional.

In addition, in another embodiment, the fertility enhancing recommendations 140 generated by the recommendation system 104 may include specific recommendations for a product. For example, the recommendation system 104 may access a database containing information on a variety of supplements in the market. Then, based on its own analysis or through the use of third party research, the recommendation system 104 may analyze the different options for a specific supplement, such as Vitamin D, to determine that a specific supplement from a first brand, Brand A, is the most beneficial supplement as compared to other Vitamin D supplements available from a second, third, and fourth brand. Such an analysis may be conducted based on the quality of the supplements, the cost of the supplements, known side effects, method of manufacture, or any other factors that may distinguish the supplement provided by one brand from a supplement provided by another brand. The recommendation system 104 may provide similar recommendations as related to food items, such as a particular type or brand of apple, and any other category of product which may require the user to select one of multiple available options.

### Supplements

In some embodiments of the invention, the recommendation system recommends specific dietary supplements for individuals with low testosterone who would like to improve their fertility.

In preferred embodiments of the invention, the dietary supplements are selected from the group of:
(i) Eurycoma longfolia;
(ii) Trigonella foenum graecum seed extract;
(iii) minerals selected from the group of: magnesium, zinc, calcium;
(iv) vitamins selected from the group of: Vitamin D;
(v) steroids selected from the group of: androstenedione, testosterone; and
(vi) tetrahydrobiopterine.

### Eurycoma Longfolia

Eurycoma longfolia is also known as Tongkat ali, Asak bumi or Malaysian ginseng and it is a flowering plant in the family Simaroubaceae. In particular, the roots of the plant are used in the formulation of the dietary supplements and beverages.

### Trigonella Foenum Graecum Seed Extract

Trigonella foenum-graecum is also known as fenugreek and it is from a plant of Fabaceae family. Fenugreek is often added as a spice in cooking.

### Magnesium

Magnesium is a mineral that is important for normal bone structure in the body. Magnesium can be sourced typically from a normal diet, but sometimes magnesium supplements are needed if magnesium levels are too low. Common dietary sources of magnesium include greens, nuts, seeds, dry beans, whole grains and low-fat dairy products.

### Zinc

Zinc is a mineral also known as an “essential trace element” because very small amounts of zinc are necessary for human health. Since the body does not store excess zinc, it must be consumed regularly as part of the diet. Common dietary sources of zinc include red meat, poultry, and fish.

### Calcium

Calcium is a mineral which is stored in bones and teeth and it is used to help muscles and blood vessels contract and expand, and for neurotransmission. Calcium is also used to help release hormones and enzymes that affect almost every function in the human body. Calcium-rich foods include milk and dairy products, kale and broccoli, as well as the calcium-enriched citrus juices, mineral water, canned fish with bones, and soy products processed with calcium.

### Vitamin D

Vitamin D is required for the regulation of the minerals calcium and phosphorus found in the body. It also plays an important role in maintaining proper bone structure. Sun exposure is an easy, reliable way for most people to get vitamin D. Vitamin D rich foods include fatty fish, such as tuna, mackerel, and salmon; liver, cheese, and egg yolks. As well as some foods fortified with vitamin D, like some dairy products, orange juice, soy milk, and cereals.

### Androstenedione

Androstenedione, or 4-androstenedione, also known as androst-4-ene-3,17-dione, is an endogenous weak androgen steroid hormone and intermediate in the biosynthesis of estrone and of testosterone from dehydroepiandrosterone. It is closely related to androstenediol.

Androstenedione is a precursor of testosterone and other androgens, as well as of estrogens like estrone, in the body. In addition to functioning as an endogenous prohormone, androstenedione also has weak androgenic activity in its own right.

### Testosterone

Testosterone is a steroid from the androstane class which is secreted primarily by the testicles of males. As previously mentioned, TRT involves high doses of testosterone which of up to about 250 mg testosterone enanthate every four weeks. Doses of testosterone at this high level may result in negative impact for fertility and conception.

However, testosterone at low levels such as 5 mg per day for between 7 to 28 days may be necessary to stimulate sperm production.

### Tetrahydrobiopterine

Tetrahydrobiopterin (BH4, THB), also known as sapropterin (INN), is a cofactor of the three aromatic amino acid hydroxylase enzymes,[4] used in the degradation of amino acid phenylalanine and in the biosynthesis of the neurotransmitters serotonin (5-hydroxytryptamine, 5-HT), melatonin, dopamine, norepinephrine (noradrenaline), epinephrine (adrenaline), and is a cofactor for the production of nitric oxide (NO) by the nitric oxide synthases. Tetrahydrobiopterin is available as a tablet for oral administration in the form of sapropterin dihydrochloride.

In a preferred embodiment of the invention, the recommendation for supplements is to be administered as separate supplements or in combination selected from the group consisting of:
(i) Eurycoma longfolia in the amount of at least about 400 mg/day for 5 weeks;
(ii) Trigonella foenum graecum seed extract in the amount of at least about 600 mg/day for 12 weeks;
(iii) minerals selected from the group of:

magnesium in the amount of at least about 10 mg/kg body weight per day preferably combined with 90-120 minutes of exercise for 4 weeks;

zinc in the amount of at least about of 25-50 mg/day for 6 months;

calcium in the amount of at least about 1500 mg/day;
(iv) vitamins selected from the group of: Vitamin D in the amount from at least about 1000 IU to 6000 IU per day
(v) steroids selected from the group of: androstenedione in the amount of at least about 300 mg/day for between 7 to 28 days, testosterone in the amount of at least about 5 mg/day for between 7 to 28 days;
(vi) tetrahydrobiopterin in the amount of at least about 5 mg/kg body weight per day.

In several embodiments of the invention, the recommendation system recommends specific supplements combined with food items for individuals with low testosterone who would like to improve their fertility. In one embodiment, the recommendation system recommends tetrahydrobiopterin may be combined with monosaturated fatty acids.

### Food Items

In some embodiments of the invention, the recommendation system recommends specific food items for individuals with low testosterone who would like to improve their fertility.

In some embodiments of the invention, the recommendation system recommends specific food items for individuals with low testosterone who would like to improve their fertility selected from the group of monounsaturated fatty acids such as virgin argan oil or extra virgin olive oil.

### Monounsatuated Fatty Acids

Monounsaturated fatty acids are fatty acids that have one double bond in the fatty acid chain with all of the remainder carbon atoms being single-bonded. Foods that are high in monounsaturated fatty acids are: avocados, almonds, cashews and peanuts.

Cooking oils made from plants or seeds such as argan oil or olive oil are sources of monosaturated fatty acids.

In a preferred embodiment of the invention, extra virgin argan oil or extra virgin olive oil are recommended food items for individuals with low testosterone who would like to improve their fertility. The recommended amount of extra virgin argan oil or extra virgin olive oil is at least about 25 ml/day.

### Onion Extract with Cysteine Sulfoxides

Onion extract is a yellow tan powder made from onions with hypoglycemic properties and the capacity to decrease platelet aggregation. Studies have shown that onion juice or extract have anti-parasitic, anti-fungal and anti-bacterial properties.

Cysteine sulfoxides are odourless, non-protein sulfur amino acids typically found in members of the family Alliaceae and are the precursors to the lachrymatory and flavour compounds found in the agronomically important genus Allium. Traditionally, Allium species, particularly the onion (Allium cepa) and garlic (A. sativum). The recommended amount of onion extract is at least about 30 mg/day.

In a preferred embodiment of the invention, onion extract with cysteine sulfoxides are recommended food items for individuals with low testosterone who would like to improve their fertility.

In several embodiments of the invention, the recommendation system recommends specific supplements combined with food items for individuals with low testosterone who would like to improve their fertility.

In one preferred embodiment, the recommendation system recommends tetrahydrobiopterin may be combined with monosaturated fatty acids.

### Specific Diet and Lifestyle Changes

In some embodiments of the invention, the recommendation system recommends specific diets or lifestyle changes for individuals with low testosterone who would like to improve their fertility.

In one embodiment of the invention, the recommendation system recommends the specific diet to follow is a vegetarian diet high in isoflavones. Isoflavones may be found in food items such as soy beans, chickpeas or pistachios.

In another embodiment of the invention, the recommendation system recommends the specific diet to follow is a ketogenic diet combined together with exercise, in particular, muscular resistance training. For example, 90-120 min/day for 5 days a week for at least 4 weeks.

In another embodiment of the invention, the recommendation system recommends specific mineral supplementation combined together with exercise. For example, magnesium supplementation in the amount of 10 mg/kg body weight per day combined with at least 90 to 120 minutes of exercise per day for 5 days a week for at least 4 weeks.

In a further embodiment of the invention, the recommendation system recommends lifestyle changes to reduce alcohol consumption to less than one drink per day.

In a further embodiment of the invention, the recommendation system recommends lifestyle changes to reduce sugary beverages to less than one drink per day.

In a further embodiment of the invention, the recommendation system recommends lifestyle changes to individuals with a BMI over 25 kg/m2 to reduce caloric intake per day, in order to lose weight.

In several embodiments of the invention, the dietary recommendations of the present invention may contribute to improving low testosterone-related symptoms such as decreased sperm production, reduced libido and erectile dysfunction.

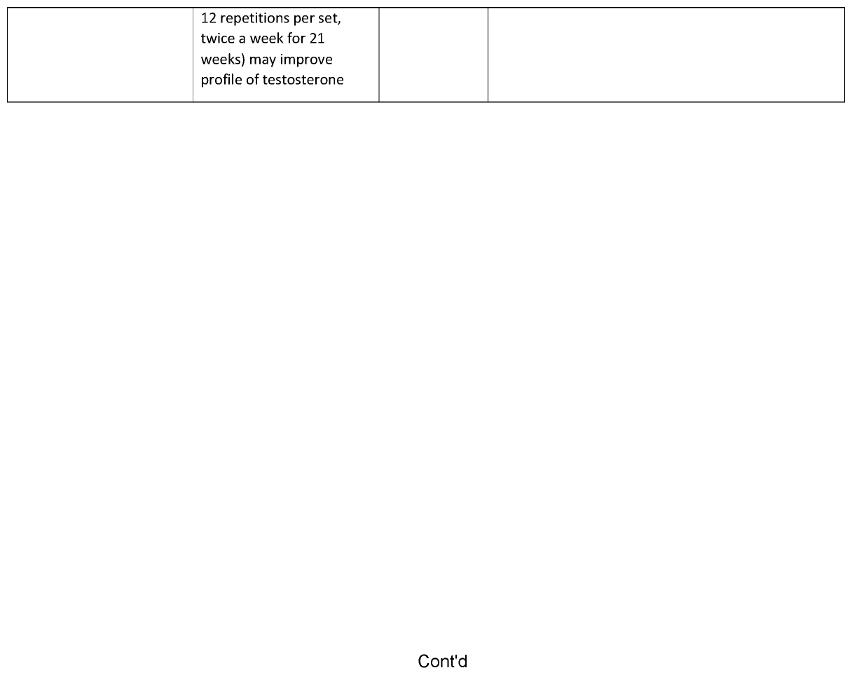

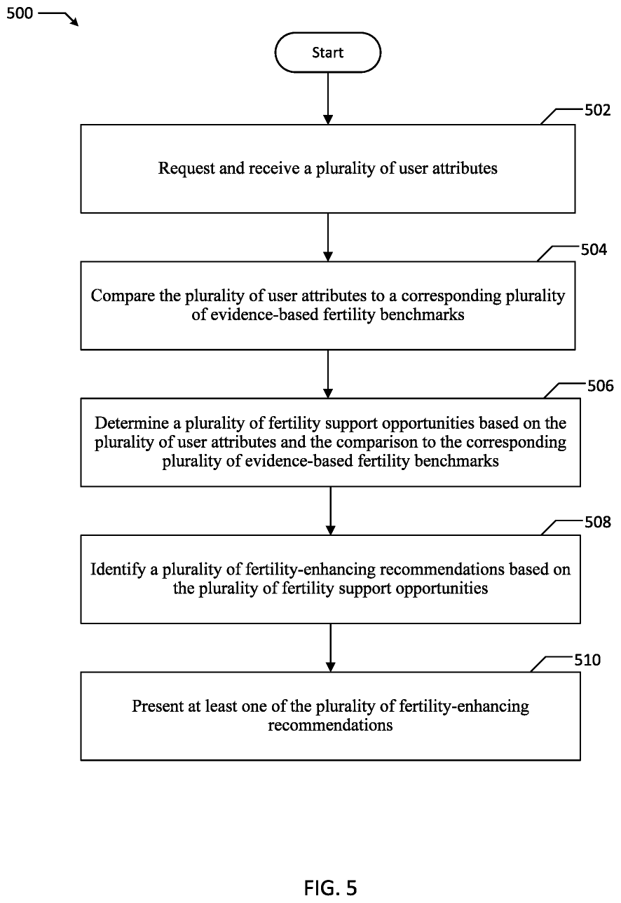

FIG. 5 illustrates an example embodiment of a method 500 of the presently disclosed method, as was discussed above in relation to the system 100. The method 500 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 500 may also be implemented by a set of instructions stored on a computer readable medium that, when executed by a processor, cause the computer system to perform the method. For example, all or part of the method 500 may be implemented by the CPU 120 and memory 118. Although the examples below are described with reference to the flowchart illustrated in FIG. 5, many other methods of performing the acts associated with FIG. 5 may be used. For example, the order of some of the blocks may be changed, certain blocks may be combined with other blocks, one or more of the blocks may be repeated, and some of the blocks described may be optional.

Block 502 can include requesting and receiving a plurality of user attributes 122. For example, a display 106 may present an attribute questionnaire 124 to solicit answers 126, to which the user device 102 provides the answers 126 to then be selected as user attributes 122. In block 504, comparisons of the plurality of user attributes 122 to a corresponding plurality of evidence-based fertility benchmarks 128 may occur. Based on these comparisons, at block 506, a plurality of fertility support opportunities 138 can be determined based on the plurality of user attributes 122 and the comparison to the corresponding plurality of evidence-based fertility benchmarks 128. At block 508, an embodiment of method 500 may identify a plurality of fertility enhancing recommendations 140 based on the plurality of fertility support opportunities 138. For example, the evidence-based diet and lifestyle recommendation engine 112 may comprise a cloud-based system trained to interpret fertility support opportunities to provide recommendations 140. Lastly, at block 510, at least one of the plurality of fertility enhancing recommendations 140 can be presented.

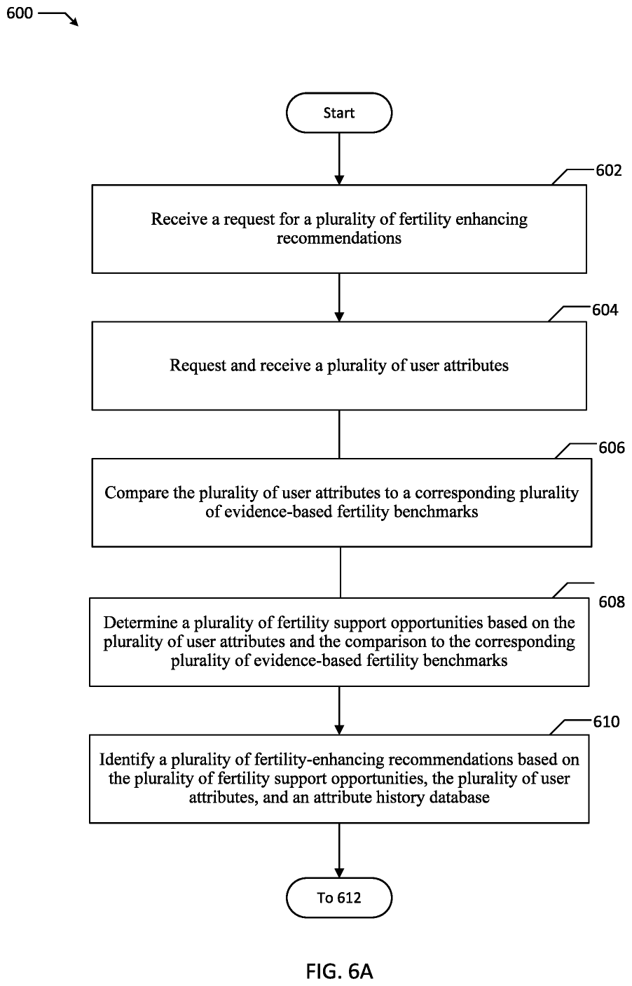

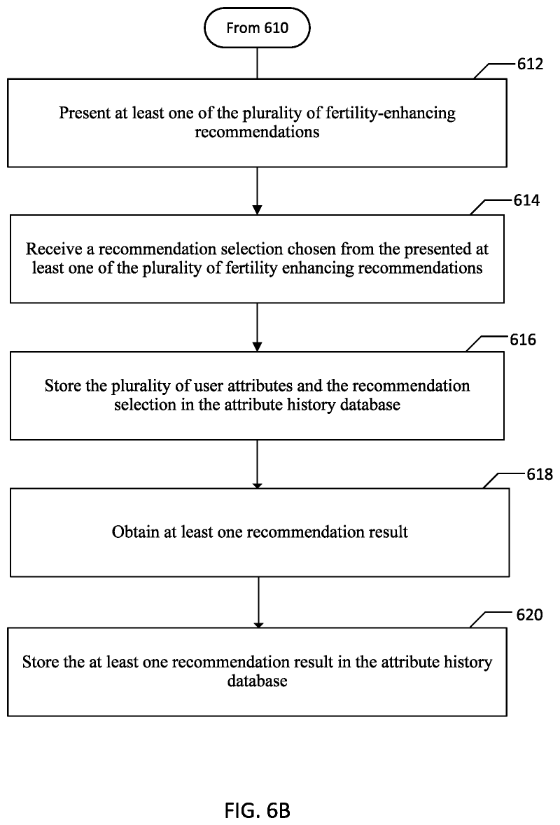

FIGS. 6A and 6B disclose an exemplary embodiment of a method 600 of the presently disclosed method. The method 600 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 600 may also be implemented by a set of instructions stored on a computer readable medium that, when executed by a processor, cause the computer system to perform the method. For example, all or part of the method 600 may be implemented by the CPU 120 and memory 118. Although the examples below are described with reference to the flowchart illustrated in FIG. 6, many other methods of performing the acts associated with FIG. 6 may be used. For example, the order of some of the blocks may be changed, certain blocks may be combined with other blocks, one or more of the blocks may be repeated, and some of the blocks described may be optional.

Block 602 may include receiving a request for a plurality of fertility enhancing recommendations 140. For example, a user may submit a request for the fertility enhancing recommendations 140 through any number of methods, including: opening an application on the user device 102, making a formal request through an application on the user device 102, submitting a request for periodic fertility enhancing recommendations 140 through the user device 102, signing into an online account through a web browser, making a formal request through a web browser, or submitting a request for periodic fertility enhancing recommendations 140 through the web browser.

At block 604, the recommendation system 104 may request and receive a plurality of user attributes 122. For example, the recommendation system 104 may present an attribute questionnaire 124 to the user. This attribute questionnaire 124 may be a standard questionnaire or a questionnaire that is customized based on known preliminary attributes, or answers to prior questions. In another example, the recommendation system 104 may request the plurality of user attributes 122 by providing a list of available home test kits, that a user may use at home. Then, after the test has been performed, the recommendation system 104 may receive the results from the test and, based on these results, determine the user attributes 122 related to such a test. For example, the home test kit may be an application to track the timing of the ovulation cycle of the user to determine the best dates for conception which may be monitored by a further application on an additional user device.

In another example, at block 604, the recommendation system 104 may provide a self-assessment tool. Similar to the prior example, the user will may make use of this self-assessment tool, submitting the results to the recommendation system 104. Again, based on the received results, the recommendation system 104 may determine the user attributes 122 based on the test. In yet another example, the recommendation system 104 may request the user have a standardized health test performed by a medical professional. In this example, the results of this performed health test may be submitted to the recommendation system 104, which thereby determines the user attributes 122 based on the results. Though some specific examples as to external tests have been given, these examples are non-limiting as the recommendation system 104 may be configured to receive results of any external or third party test in order to determine the corresponding user attributes 122.

In Block 606, the recommendation system 104 may be configured to compare the plurality of user attributes 122 to a corresponding plurality of evidence-based fertility benchmarks 128. For example, these evidence-based fertility benchmarks 128 may include standardized benchmarks, as in benchmarks that are given to all, regardless of individual variances. In another example, these benchmarks 128 may be customized based on a particular user's history or goals. For example, if a healthy user is trying to improve his or her fertility and the current user attributes 122 exceeds all standard evidence-based fertility benchmarks 128, the recommendation system 104 may be configured to determine a customized fertility benchmark set 132 for which the particular user should aim. In contrast, in another example, a different user that is far below a standard evidence-based fertility benchmark 128 may be compared to a different, lower benchmark value as a manner of inspiring progress and providing milestones.

The example method, at Block 608, may be configured to determine a plurality of fertility support opportunities 138 based on the plurality of user attributes 122 and the comparison to the corresponding plurality of evidence-based fertility benchmarks 128. For example, the recommendation system 104 may determine that a user attribute 122 corresponds to an above optimal stress level. Based on this comparison, the recommendation system 104 may determine a fertility support opportunity 138 to reduce stress. In another example, the recommendation system 104 may determine that the user has not yet seen a medical professional, and, as such, determine a fertility support opportunity 138 to visit a medical professional.

At Block 610, the recommendation system 104 may identify a plurality of fertility-enhancing recommendations 140 based on at least the plurality of fertility support opportunities 138. For example, the recommendation system may determine a plurality of similar prior cases by analyzing the attribute history database 148, identifying similarities between the user attributes 122 received and the plurality of prior user attributes within the attribute history database 148. For example, the recommendation system 104 may identify that the user attributes 122 detail a user with an above average BMI and other similarities that correspond to a particular group of past users and therefore the cases of those member of that particular group of past users are determined as similar prior cases.

Furthermore, in this example, the recommendation system 104 may determine a plurality of prior case results based on the plurality of similar prior cases. As detailed previously, the attribute history database 148 may comprise corresponding recommendations associated with prior user attributes, and the effectiveness of these corresponding recommendations. As such, the recommendation system 104 may analyze the corresponding recommendations and their effectiveness as associated with the particular group of past users to determine a plurality of prior case results.

Furthermore, in this example, the recommendation system 104 may determine successful recommendations and a plurality of unsuccessful recommendations based on a plurality of prior case results. For example, the recommendation system 104 may have recommended the users in that particular group of past users increase exercise levels in some cases and decrease food consumption in other cases. Based on prior case results as determined based on the attribute history database 148, the recommendation system 104 may determine that the recommendations for decreasing food consumption were not very successful, yet increasing exercise levels proved to be very successful and, as such, determines that increasing exercise levels is a successful recommendation whereas decreasing food consumption is an unsuccessful recommendation. By conducting analysis of these prior user attributes, the recommendation selection and the effectiveness of corresponding recommendations, the recommendation system 104 may identify trends associated with different subset patient populations, thereby creating and validating a plurality of lifestyle interventions. These examples of successful and unsuccessful recommendations are nonlimiting, as different groups may experience different levels of success to the same recommendations.

In addition, the recommendation system 104 may be configured to determine a plurality of fertility enhancing recommendations based on the plurality of successful recommendations and the plurality of unsuccessful recommendations. For example, the recommendation system 104 may be configured to only recommend the plurality of successful recommendations. In another example, the recommendation system 104 may still recommend any of the unsuccessful recommendations. The recommendation system 104 may make these recommendations based on any number of reasons, including a slight difference in the user attributes 122 as compared to the prior user attributes, a lack of insufficient data to support a true unsuccessful recommendation, or data supporting that, although unsuccessful, the recommendation is popular and often followed through by users. In another example, the recommendation system 104 may recommend less than all of the plurality of successful recommendations. In an example, the decision for selecting which of the plurality of recommendations to present generate may be performed by an Al.

In another example, successful recommendations may be based on guidelines associated with particular medical conditions, such as a user with low testosterone. In that case, these guidelines would be determined as successful recommendations.

At Block 612, the recommendation system may present at least one of the plurality of fertility-enhancing recommendations 140. At Block 614, the recommendation system 104 may receive a recommendation selection chosen from the presented at least one of the plurality of fertility enhancing recommendations 140. For example, a user may be presented with three fertility-enhancing recommendations 140, to follow a ketogenic diet; to increase consumption of monosaturated fats; and to reduce alcohol consumption to less than one drink per day. The user may select one, two, or all three of these options. As such, the recommendation system 104 receives, from the user device 102, these two selected recommendations as the recommendation selection. In another example, the user may not select any of the presented recommendations, at which point the recommendation system 104 may generate and present a different plurality of fertility enhancing recommendations 140.

In another example, after the user reviews the presented fertility enhancing recommendations 140, the user may submit a request to contact a fertility coach. For example, the user may be undecided as to how to implement the recommendations or may simply have a question for which the user seeks an answer. In some instances, the recommendation system 104 may determine that the question for which the user seeks an answer may be adequately answered by a virtual coach, and thereby provides access to and interaction with said virtual coach. In other instances, the recommendation system 104 may determine that the question will be best handled by a personal coach, a living individual, and thereby provides access to and interaction with said personal coach.

At Block 616, the recommendation system 104 may store the plurality of user attributes 122 and the recommendation selection in the attribute history database 148. For example, the recommendation system 104 may store all user attributes 122 received on a first day, along with the recommendation selection received on that same first day. These user attributes 122 and recommendation selection may then be accessed by the recommendation system 104 in the future when analyzing the attribute history database 148.

The recommendation system 104 may obtain at least one recommendation result at Block 618. In an example, the user may submit a recommendation result through the user device 102. This result may include a qualitative or quantitative rating as selected by the user. In another example, the recommendation system 104 may receive a future plurality of user attributes 122 and, at that time, compare the received future user attributes with the previously received user attributes, now prior user attributes, within the attribute history database 148. Based on this comparison, the recommendation system 104 may determine a recommendation result, such as decreased or increased BMI. After obtaining this recommendation result, the recommendation system 104 may store the at least one recommendation result in the attribute history database 148, corresponding to the prior recommendation selection. This recommendation system 104 may then await another request for fertility enhancing recommendations 140, and at that time, perform the method 600 again at Block 602.

Such an example method as disclosed in FIGS. 6A and 6B allow for the continuous, customized, integrated recommendation system 104 to endlessly improve upon recommendations as the attribute history database 148 grows in size. Through this growth, the recommendation system 104, and in some embodiments, the evidence-based diet and lifestyle recommendation engine 112, will have an ever-expanding set of data from which it can derive fertility enhancing recommendations 140, with increasing particularity regarding what users receive which recommendations.

In another aspect, a method of treatment may comprise using any of the above described systems or methods to generate any one or more of the fertility enhancing recommendations 140, diet and lifestyle recommendations, or specific supplementation recommendations. Furthermore, the method of treatment may comprise administering a treatment based on at least the any of one or more of the fertility enhancing recommendations 140, diet and lifestyle recommendations, or specific supplementation recommendations to a user. For example, when the recommendation system 104 determines a fertility enhancing recommendation 140 that comprises increasing a user's vitamin D intake from 1000 mg/day to 6000 mg/day by way of a 5000 mg vitamin D supplement, an example method of treatment may comprise administering a treatment comprising a 5000 mg vitamin D supplement to the user each day.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1: Dietary Recommendations for Individuals with Low Testosterone

The following are dietary recommendations or supplements for individual with low testosterone or hypogonadism.

| Diet and | | Supplements/ | | |
|---|---|---|---|---|
| lifestyle | Dietary | Diet/ | | |
| components | recommendation | Lifestyle | Tips for the consumer | Ref. |
| Virgin Argan Oil | 25 ml/day for at | Diet | VAO and EVO may | [1] |
| (VAO) | least 3 weeks | | stimulate T biosynthesis by | |
| Extra Virgin | | | a mechanism that could | |
| Olive Oil (EVO) | | | involve an activation of the | |
| | | | hypothalamo-pituitary- | |
| | | | testicular axis and/or an | |
| | | | induction of steroidogenic | |
| | | | proteins. | |
| Herbs | Eurycoma | Supplement | Men may benefit from | [2, 3] |
| | longifolia (400 mg/ | | taking Eurycoma longfolia | |
| | day for at least 5 | | or Trigonella Foenum- | |
| | weeks) | | graecum seed extract for | |
| | Trigonella | | increasing testosterone | |
| | foenum-graecum | | levels | |
| | seed extract (600 mg/ | | | |
| | day for at | | | |
| | least 12 weeks) | | | |
| Isoflavones | | Diet | Young vegetarian men (18- | [4] |
| (vegetarian | | | 34 years) show higher | |
| diet) | | | levels of testosterone with | |
| | | | vegetarian diet high in | |
| | | | isoflavones | |
| Magnesium | 10 mg/kg body | Supplement + | Supplementation with | [5] |
| (MgSO₄) and | weight/day + 90- | Lifestyle | magnesium increases free | |
| exercise | 120 min/day (5 | | and total testosterone | |
| | days a week) for | | values in sedentary | |
| | at least 4 weeks | | individuals and athletes. | |
| | | | The increases are higher in | |
| | | | those who exercise than in | |
| | | | sedentary individuals. | |
| Zinc | Zinc | Supplement | Zinc may improve | [6, 7] |
| | supplementation | | testosterone levels in | |
| | (25-50 mg/day | | healthy men but also in | |
| | for at least 6 | | men with chronic kidney | |
| | months) | | disease and in men with | |
| | | | isolated hypogonadotropic | |
| | | | hypogonadism that are | |
| | | | treated with sequential | |
| | | | purified urinary follicular- | |
| | | | stimulating | |
| | | | hormone/human chorionic | |
| | | | gonadotropin (uFSH/hCG) | |
| | | | replacement | |
| Combination of | Vitamin D (1,000 | Diet and | Calcium and vitamin D may | [8] |
| Vitamin D, | IU/day), calcium | Supplement | be helpful as adjuvant to | |
| Calcium and | (1500 mg/day, | | oral testosterone for | |
| oral | testosterone (5 | | increasing testosterone | |
| testosterone | mg/day) | | levels | |
| Vitamin D | High dose vitamin | Supplement | Oral supplementation of | [9] |
| | D supplement in | | high Vitamin D may help to | |
| | vitamin D | | increase testosterone | |
| | deficient males | | levels in men that are | |
| | (6000 IU vitamin | | vitamin D deficient | |
| | D initially and | | | |
| | later the same | | | |
| | dose once per | | | |
| | month, every | | | |
| | other month or | | | |
| | every 3 months | | | |
| | for 1 year) | | | |
| Onion extract | 30 mg/day | Supplement | Salivary testosterone was | [10] |
| containing | | | significantly higher after 14 | |
| concentrated | | | days of supplements | |
| cysteine | | | administration | |
| sulfoxides | | | | |
| Ketogenic diet | 2600 kcal, 217 g | Diet and | The ketogenic diet can be | [11] |
| and exercise | of fat, 31 g of | lifestyle | used in combination with | |
| | carbohydrates, | | resistance training to cause | |
| | 17 g of fiber, 134 g | | favorable changes in | |
| | of protein, 10 | | hormonal profiles in | |
| | weeks | | resistance-trained males | |
| Androstenedione | 300 mg/day for 7- | Supplement | Short-term | [12, 13] |
| | 28 days | | androstenedione | |
| | | | supplementation increased | |
| | | | acute testosterone | |
| | | | responses to resistance | |
| | | | exercise in older men | |
| Healthy diet | Healthy diet | Diet | A healthy dietary pattern, | [14-16] |
| | pattern | | such as avoiding a high | |
| | Magnesium: 350 | | consumption of bread and | |
| | mg/day* | | pastries, dairy products, | |
| | Iron: 11 mg/day* | | and desserts, eating out, | |
| | Zinc: 9.4-16.3 | | alcohol, and a high intake | |
| | mg/day* | | of homemade foods, | |
| | | | noodles, and dark green | |
| | | | vegetables may be | |
| | | | associated with improved | |
| | | | testosterone levels | |
| | | | Especially magnesium, iron | |
| | | | and zinc may be beneficial | |
| | | | for normal testosterone | |
| | | | levels | |
| Diet | 6335 kcal over 42 | Diet | Men in extreme climate | [17-19] |
| | days | | (polar expedition) or | |
| | | | extreme exercise should | |
| | | | ensure sufficient energy | |
| | | | intake to avoid decrease in | |
| | | | testosterone | |
| Dietary fat and | Single | Diet and | Taking BH4 alongside a | [20] |
| supplement | administration of | Supplement | high fat meal could | |
| | a high fat meal | | improve the profile of | |
| | (48 g; 46 | | testosterone in men | |
| | energy %) in | | | |
| | combination with | | | |
| | tetrahydrobiopterin, | | | |
| | BH4 (5 mg/kg | | | |
| | body weight) | | | |
| Exercise | Normocaloric diet | Lifestyle | Exercising while keeping a | [21] |
| | and mild exercise | | weight maintaining diet | |
| | adjunctive to 850 | | may help to improve low | |
| | mg metformin | | testosterone in men with | |
| | twice daily, over | | metabolic syndrome | |
| | 4 months | | | |
| Alcohol | <40 g/day | Diet | Men with low testosterone | [22] |
| | | | may benefit from | |
| | | | consuming less than 40 g | |
| | | | alcohol per day | |
| Sugar | ≤137 kcal/day | Diet | SSB consumption is | [23] |
| sweetened | | | significantly associated | |
| beverages (SSB) | | | with low serum | |
| | | | testosterone in men 20-39 | |
| | | | years old in the US. Men | |
| | | | may benefit from | |
| | | | consuming SSB in amounts | |
| | | | less than 137 kcal per day | |
| BMI | BMI <25 kg/m2 | Lifestyle | BMI ≥25 kg/m2 had a | [23] |
| | | | higher risk of having a low | |
| | | | testosterone level than | |
| | | | those with a BMI <25 kg/m2 | |
| Caloric | Reduced calorie | Lifestyle | Profiles of testosterone in | [16, |
| restriction and | e.g. 1200 kcal or | | overweight may improve | 24-33] |
| exercise for | very low calorie | | with exercise/weight loss | |
| obese men | diets (either | | and subsequent weight | |
| | through diet or | | maintenance | |
| | meal | | | |
| | replacements) | | | |
| | ranging from for | | | |
| | 6 to 24 months | | | |
| | with or without | | | |
| | physical activity. | | | |
| | Physical activity | | | |
| | includes | | | |
| | moderate aerobic | | | |
| | exercise (50-70% | | | |
| | max heart rate) | | | |
| | for 150 min per | | | |
| | week to high | | | |
| | intensity aerobic | | | |
| | exercise (>70% | | | |
| | max heart rate) | | | |
| | for 60 min per | | | |
| | day. | | | |
| | Energy deficit | | | |
| | ranged from 170 | | | |
| | kcal per day | | | |
| | to 40% | | | |
| | of daily energy | | | |
| | requirements. | | | |
| Caloric | Calorie-reduced | Lifestyle | Exercise and a moderate | [34] |
| restriction and | diet (400 kcal | | calorie reduced diet may | |
| exercise for | below calculated | | help to improve low | |
| normal weight | requirement) or | | testosterone in normal | |
| men | increased | | weight men | |
| | carbohydrate and | | | |
| | reduced fat in | | | |
| | combination with | | | |
| | strength exercise | | | |
| | (40% to 80% of | | | |
| | maximum load, 3- | | | |
| | 6 sets per | | | |
| | exercise, with 8- | | | |
| | 12 repetitions per | | | |
| | set, twice a week | | | |
| | for 21 weeks) | | | |
| | may improve | | | |
| | profile of | | | |
| | testosterone | | | |

*EFSA, Dietary Reference Values (DRV) 2017

### REFERENCES

1. Derouiche, A., et al., Effect of argan and olive oil consumption on the hormonal profile of androgens among healthy adult Moroccan men. Nat Prod Commun, 2013. 8(1): p. 51-3.
2. Henkel, R. R., et al., Tongkat Ali as a potential herbal supplement for physically active male and female seniors—a pilot study. Phytother Res, 2014. 28(4): p. 544-50.
3. Rao, A., et al., Testofen, a specialised Trigonella foenum-graecum seed extract reduces age-related symptoms of androgen decrease, increases testosterone levels and improves sexual function in healthy aging males in a double-blind randomised clinical study. Aging Male, 2016. 19(2): p. 134-42.
4. Hod, R., et al., Plasma isoflavones in Malaysian men according to vegetarianism and by age. Asia Pac J Clin Nutr, 2016. 25(1): p. 89-96.
5. Cinar, V., et al., Effects of magnesium supplementation on testosterone levels of athletes and sedentary subjects at rest and after exhaustion. Biol Trace Elem Res, 2011. 140(1): p. 18-23.
6. Chang, C. S., et al., Correlation between serum testosterone level and concentrations of copper and zinc in hair tissue. Biol Trace Elem Res, 2011. 144(1-3): p. 264-71.
7. Vecchio, M., et al., Treatment options for sexual dysfunction in patients with chronic kidney disease: a systematic review of randomized controlled trials. Clin J Am Soc Nephrol, 2010. 5(6): p. 985-95.
8. Kenny, A. M., et al., Effects of transdermal testosterone on bone and muscle in older men with low bioavailable testosterone levels, low bone mass, and physical frailty. J Am Geriatr Soc, 2010. 58(6): p. 1134-43.
9. Canguven, O., et al., Vitamin D treatment improves levels of sexual hormones, metabolic parameters and erectile function in middle-aged vitamin D deficient men. Aging Male, 2017. 20(1): p. 9-16.
10. Nakayama, Y., et al., Alleviation of the aging males' symptoms by the intake of onion-extracts containing concentrated cysteine sulfoxides for 4 weeks-randomized, double-blind, placebo-controlled, parallel-group comparative study. Japanese Pharmacology and Therapeutics, 2017. 45: p. 595-608.
11. Wilson, J. M., et al., The Effects of Ketogenic Dieting on Body Composition, Strength, Power, and Hormonal Profiles in Resistance Training Males. J Strength Cond Res, 2017.
12. Brown, G. A., et al., Effects of androstenedione-herbal supplementation on serum sex hormone concentrations in 30- to 59-year-old men. Int J Vitam Nutr Res, 2001. 71(5): p. 293-301.
13. Judge, L. W., et al., Effects of acute androstenedione supplementation on testosterone levels in older men. Aging Male, 2016. 19(3): p. 161-167.
14. Hu, T. Y., et al., Testosterone-Associated Dietary Pattern Predicts Low Testosterone Levels and Hypogonadism. Nutrients, 2018. 10(11).
15. Rotter, I., et al., Analysis of the relationship between the blood concentration of several metals, macro- and micronutrients and endocrine disorders associated with male aging. Environ Geochem Health, 2016. 38(3): p. 749-61.
16. Armamento-Villareal, R., et al., Effect of Lifestyle Intervention on the Hormonal Profile of Frail, Obese Older Men. J Nutr Health Aging, 2016. 20(3): p. 334-40.
17. Geesmann, B., et al., Association Between Energy Balance and Metabolic Hormone Suppression During Ultraendurance Exercise. Int J Sports Physiol Perform, 2017. 12(7): p. 984-989.
18. Hooper, D. R., et al., The presence of symptoms of testosterone deficiency in the exercise-hypogonadal male condition and the role of nutrition. Eur J Appl Physiol, 2017. 117(7): p. 1349-1357.
19. Woods, D. R., et al., Nutritional status and the gonadotrophic response to a polar expedition. Appl Physiol Nutr Metab, 2015. 40(3): p. 292-7.
20. Shah, Y., et al., BH4 improves postprandial endothelial function after a high-fat meal in men and postmenopausal women. Menopause, 2017. 24(5): p. 555-562.
21. Casulari, L. A., et al., Effects of metformin and short-term lifestyle modification on the improvement of male hypogonadism associated with metabolic syndrome. Minerva Endocrinol, 2010. 35(3): p. 145-51.
22. Sierksma, A., et al., Effect of moderate alcohol consumption on plasma dehydroepiandrosterone sulfate, testosterone, and estradiol levels in middle-aged men and postmenopausal women: a diet-controlled intervention study. Alcohol Clin Exp Res, 2004. 28(5): p. 780-5.
23. Chen, L., et al., Sugar-sweetened beverage intake and serum testosterone levels in adult males 20-39 years old in the United States. Reprod Biol Endocrinol, 2018. 16(1): p. 61.
24. De Lorenzo, A., et al., MOSH Syndrome (Male Obesity Secondary Hypogonadism): Clinical Assessment and Possible Therapeutic Approaches. Nutrients, 2018. 10(4).
25. Khoo, J., et al., Comparing the effects of meal replacements with reduced-fat diet on weight, sexual and endothelial function, testosterone and quality of life in obese Asian men. Int J Impot Res, 2014. 26(2): p. 61-6.
26. Khoo, J., et al., Effects of a low-energy diet on sexual function and lower urinary tract symptoms in obese men. Int J Obes (Lond), 2010. 34(9): p. 1396-403.
27. Khoo, J., et al., Comparing effects of low- and high-volume moderate-intensity exercise on sexual function and testosterone in obese men. J Sex Med, 2013. 10(7): p. 1823-32.
28. Kumagai, H., et al., Lifestyle modification increases serum testosterone level and decrease central blood pressure in overweight and obese men. Endocr J, 2015. 62(5): p. 423-30.
29. Lima, N., et al., Decreased androgen levels in massively obese men may be associated with impaired function of the gonadostat. Int J Obes Relat Metab Disord, 2000. 24(11): p. 1433-7.
30. Moran, L. J., et al., Long-Term Effects of a Randomised Controlled Trial Comparing High Protein or High Carbohydrate Weight Loss Diets on Testosterone, SHBG, Erectile and Urinary Function in Overweight and Obese Men. PLoS One, 2016. 11(9): p. e0161297.
31. Niskanen, L., et al., Changes in sex hormone-binding globulin and testosterone during weight loss and weight maintenance in abdominally obese men with the metabolic syndrome. Diabetes Obes Metab, 2004. 6(3): p. 208-15.
32. Reis, L. O., et al., Bariatric surgery does not interfere with sperm quality—a preliminary long-term study. Reprod Sci, 2012. 19(10): p. 1057-62.
33. Schulte, D. M., et al., Caloric restriction increases serum testosterone concentrations in obese male subjects by two distinct mechanisms. Horm Metab Res, 2014. 46(4): p. 283-6.
34. Sallinen, J., et al., Dietary intake, serum hormones, muscle mass and strength during strength training in 49-73-year-old men. Int J Sports Med, 2007. 28(12): p. 1070-6.
35. Zirkin et al. Aging and Declining Testosterone: Past, Present, and Hopes for the Future. J Androl. 2012 November-December; 33(6): 1111-1118.

1. A method of enhancing fertility and conception for individuals with low testosterone levels comprising:
requesting and receiving a plurality of user attributes;
comparing the plurality of user attributes to a corresponding plurality of evidence-based fertility benchmarks;
determining a plurality of fertility support opportunities based on at least the plurality of user attributes and the comparison to the corresponding plurality of evidence-based fertility benchmarks;
identifying a plurality of fertility enhancing recommendations based on at least the plurality of fertility support opportunities; and
presenting at least one of the plurality of fertility enhancing recommendations.

2. The method according to claim 1 wherein the individuals are considered to have low testosterone levels, if the testosterone levels are below 270 nanograms per deciliter (ng/dL) as measured in semen.

3. The method according to claim 1, wherein the plurality of user attributes comprises information regarding the condition of low testosterone and at least one of the user attributes selected from: age, gender, weight, height, activity level, food sensitivities, preferred diet, fertility status, lifestyle choices, or any co-morbidities.

4. The method according to claim 1 wherein the individuals are male and at least 30 years of age or older.

5. The method according to claim 1, wherein the identifying of a plurality of fertility enhancing recommendations includes the dietary recommendations.

6. The method according to any claim 1, wherein the identifying of a plurality of fertility enhancing recommendations includes recommendations for supplements.

7-8. (canceled)

9. The method according to claim 6, wherein the supplements are selected from
the group consisting of:
(i) Eurycoma longfolia in the amount of at least about 400 mg/day for 5 weeks;
(ii) Trigonella foenum graecum seed extract in the amount of at least about 600 mg/day for 12 weeks;
(iii) minerals selected from the group of:
magnesium in the amount of at least about 10 mg/kg body weight per day preferably combined with 90-120 minutes of exercise for 4 weeks;
zinc in the amount of at least about of 25-50 mg/day for 6 months;
calcium in the amount of at least about 1500 mg/day;
(iv) vitamins selected from the group of: Vitamin D in the amount from at least about 1000 IU to 6000 IU per day;
(v) steroids selected from the group of: androstenedione in the amount of at least about 300 mg/day for between 7 to 28 days, testosterone in the amount of at least about 5 mg/day for between 7 to 28 days; and
(vi) tetrahydrobiopterin in the amount of at least about 5 mg/kg body weight per day.

10. The method according to claim 9 wherein the recommendation for supplements be
administered in the combination of Vitamin D, calcium and testosterone wherein Vitamin D is in
the amount of at least about 1000 IU/day; calcium is in the amount of at least about 1500 mg/day
and testosterone is in the amount of at least about 5 mg/day.

11. The method according to claim 1, wherein the identifying of a plurality of fertility enhancing recommendations includes food recommendations selected from the group of monounsaturated fatty acids consisting of: virgin argan oil or extra virgin olive oil.

12. The method according to claim 1, wherein the identifying of a plurality of fertility enhancing recommendations includes food recommendations of consuming onion extract with cysteine sulfoxide content.

13. The method according to claim 11, wherein the identifying of a plurality of fertility enhancing recommendations includes food recommendations selected from the group of:
(i) consuming virgin argan oil in the amount of at least about 25 ml/day;
(ii) consuming extra virgin olive oil in the amount of at least about 25 mg/day; and
(iii) consuming onion extract with cysteine sulfoxide content in the amount of at least about 30 mg/day.

14. The method according to claim 1, wherein the identifying of a plurality of fertility enhancing recommendations includes diet recommendations selected from the group of:
(i) following a ketogenic diet;
(ii) following a vegetarian diet high in isoflavones;
(iii) reducing alcoholic beverages to less than one per day;
(iv) reducing sugary beverages to less than one per day; and
(iv) reducing daily energy consumption for individuals with a BMI over 25 kg/m2.

15. The method according to claim 1, wherein the identifying of a plurality of fertility enhancing recommendations includes lifestyle recommendations:
(i) to avoid testicular injury and scrotal overheating,
(ii) to avoid excessive alcohol consumption, and
(iii) to avoid chronic narcotic use and marijuana use.

16. The method according to claim 1 for preventing or treating low testosterone levels, decreased sperm production, reduced libido and/or erectile dysfunction.

17. The method according to claim 1, wherein the identifying of a plurality of fertility enhancing recommendations based on at least the plurality of fertility support opportunities comprises:
providing the plurality of fertility support opportunities to a cloud-based artificial intelligence service; and
receiving, from the cloud-based artificial intelligence service, a plurality of fertility enhancing recommendations based on the fertility support opportunities provided to the cloud-based artificial intelligence service.

18. A computer implemented system for generating fertility enhancing recommendations for enhancing fertility and conception for individuals with low testosterone comprising:
a memory;
a CPU;
a display configured to present an attribute questionnaire to a user;
an attribute receiving unit configured to receive a plurality of user attributes based on at least the attribute questionnaire;
an attribute comparison unit configured to compare the received plurality of user attributes to a corresponding plurality of fertility attribute benchmarks;
an evidence-based diet and lifestyle recommendation engine configured to generate a plurality of fertility enhancing recommendations based on at least the plurality of user attributes and the comparison to corresponding plurality of fertility attribute benchmarks; and
wherein the display is further configured to present at least one of the plurality of the fertility enhancing recommendations to the user.

19. The system of claim 18, wherein the plurality of user attributes comprises information regarding the individual with low testosterone selected from the group:
level of testosterone below 270 nanograms per deciliter (ng/dL).

20. The system according to claim 18, wherein the plurality of user attributes comprises information regarding at least one further user attribute selected from the group of: age, gender, weight, height, activity level, food sensitivities, preferred diet, fertility status, lifestyle choices, and any co-morbidities.

21. (canceled)

22. The system according to claim 18, wherein the evidence-based diet and lifestyle recommendation engine comprises:
a plurality of databases comprising one or more of recipes, specific food items, products, or diet tips;
a plurality of filters by dietary restrictions comprising one or more of food sensitivities, preferred diets, fertility-related conditions, or co-morbidities; and
an optimization unit configured to optimize the plurality of fertility enhancing recommendations based on one or more of caloric intake, food groups, or nutrients.

23. The system according to claim 18, wherein the optimization unit is configured to optimize fertility enhancing recommendations including recommendations of dietary supplements for individuals with low testosterone, comprising dietary supplements selected from the group of:
(i) Eurycoma longfolia;
(ii) Trigonella foenum graecum seed extract;
(iii) minerals selected from the group of: magnesium, zinc, calcium;
(iv) vitamins selected from the group of: Vitamin D;
(v) steroids selected from the group of: androstenedione, testosterone; and
(vi) tetrahydrobiopterine.

24. The system of claim 18, wherein the optimization unit is configured to optimize fertility enhancing recommendations including recommendations of dietary supplements for individuals with low testosterone, comprising supplements administered in the following dosing amounts as separate supplements or in combination selected from the group consisting of:
(i) Eurycoma longfolia in the amount of at least about 400 mg/day for 5 weeks;
(ii) Trigonella foenum graecum seed extract in the amount of at least about 600 mg/day for 12 weeks;
(iii) minerals selected from the group of:
magnesium in the amount of at least about 10 mg/kg body weight per day preferably combined with 90-120 minutes of exercise for 4 weeks;
zinc in the amount of at least about of 25-50 mg/day for 6 months;
calcium in the amount of at least about 1500 mg/day;
(iv) vitamins selected from the group of: Vitamin D in the amount from at least about 1000 IU to 6000 IU per day;
(v) steroids selected from the group of: androstenedione in the amount of at least about 300 mg/day for between 7 to 28 days, testosterone in the amount of at least about 5 mg/day for between 7 to 28 days; and
(vi) tetrahydrobiopterin in the amount of at least about 5 mg/kg body weight per day.

25. The system of claim 18 wherein the optimization unit is configured to optimize fertility enhancing recommendations including food recommendations for individuals with low testosterone, comprising food recommendations selected from the group of:
(i) consuming virgin argan oil in the amount of at least about 25 ml/day;
(ii) consuming extra virgin olive oil in the amount of at least about 25 mg/day; and
(iii) consuming onion extract with cysteine sulfoxide content in the amount of at least about 30 mg/day.

26-28 (canceled)
